(19) United States
(12) Patent Application Publication
(10) Pub. No.: US 2022/0084686 A1
(43) Pub. Date: Mar. 17, 2022

(54) INTELLIGENT PROCESSING OF BULK HISTORIC PATIENT DATA

(71) Applicant: International Business Machines Corporation, Armonk, NY (US)

(72) Inventors: Mark D. Bronkalla, Waukesha, WI (US); Amit Khare, Waukesha, WI (US)

(21) Appl. No.: 17/019,056

(22) Filed: Sep. 11, 2020

(51) Int. Cl. G16H 50/70; G16H 10/60; G16H 15/00; G06F 16/28; G06F 16/2455; G06N 20/00

(52) U.S. Cl. CPC G16H 50/70; G16H 10/60; G06N 20/00; G06F 16/285; G06F 16/24556; G16H 15/00

(57) ABSTRACT

Aspects of the present invention disclose a method for processing bulk historical data. The method includes one or more processors identifying one or more features of messages of incoming data queries of a computing device, wherein the one or more features include structured and unstructured data. The method further includes aggregating one or more segments of bulk historic data for a plurality of individuals based at least in part on the one or more features of the messages of the incoming data queries. The method further includes determining a classification of each individual of the plurality of individuals based at least in part on the aggregated one or more segments of the bulk historic data. The method further includes prioritizing processing of the aggregated one or more segments of the bulk historic data based at least in part on the classification of each individual of the plurality of individuals.

### BACKGROUND OF THE INVENTION

The present invention relates generally to the field of record analytics, and more particularly to historical patient medical record processing.

In computing, extract, transform, load (ETL) is the general procedure of copying data from one or more sources into a destination system which represents the data differently from the source(s) or in a different context than the source(s). ETL systems commonly integrate data from multiple applications (systems), typically developed and supported by different vendors or hosted on separate computer hardware.

Health Level Seven (HL7) refers to a set of international standards for transfer of clinical and administrative data between software applications used by various healthcare providers. Hospitals and other healthcare provider organizations typically have many different computer systems used for everything from billing records to patient tracking. Such guidelines or data standards are a set of rules that allow information to be shared and processed in a uniform and consistent manner. However, much of the medical record is based on unstructured free text such as visit notes, surgical notes, imaging reports, etc. These data standards are meant to allow healthcare organizations to easily share clinical information. An HL7 message is a hierarchical structure associated with a trigger event. The HL7 standard defines trigger event as an event in the real world of health care that creates the need for data to flow among systems. Each trigger event is associated with an abstract message that defines the type of data that the message needs to support the trigger event.

Machine learning is the scientific study of algorithms and statistical models that computer systems use to perform a specific task without using explicit instructions, relying on patterns and inference instead. Machine learning is seen as a subset of artificial intelligence. Machine learning algorithms build a mathematical model based on sample data, known as “training data,” in order to make predictions or decisions without being explicitly programmed to perform the task. Machine learning algorithms are used in a wide variety of applications.

### SUMMARY

Aspects of the present invention disclose a method, computer program product, and system for processing bulk historical data. The method includes one or more processors identifying one or more features of messages of incoming data queries of a computing device, wherein the one or more features include structured and unstructured data. The method further includes one or more processors aggregating one or more segments of bulk historic data for each individual of a plurality of individuals based at least in part on the one or more features of the messages of the incoming data queries. The method further includes one or more processors determining a classification of each individual of the plurality of individuals based at least in part on the aggregated one or more segments of the bulk historic data. The method further includes one or more processors prioritizing processing of the aggregated one or more segments of the bulk historic data based at least in part on the classification of each individual of the plurality of individuals.

### BRIEF DESCRIPTION OF THE DRAWINGS

FIG. 1 is a functional block diagram of a data processing environment, in accordance with an embodiment of the present invention.

FIG. 2 is a flowchart depicting operational steps of a program, within the data processing environment of FIG. 1, for processing bulk historical data, in accordance with embodiments of the present invention.

FIG. 3 is a block diagram of components of the client device and server of FIG. 1, in accordance with an embodiment of the present invention.

### DETAILED DESCRIPTION

Embodiments of the present invention provide algorithms for extracting medical concepts from the unstructured text of the medical records. Accordingly, embodiments of the present invention can operate to build a historical synopsis or summary based on the extracted concepts of the patient's medical records.

Embodiments of the present invention allow for queuing of bulk historical data of a patient for processing based on a determined priority. Embodiments of the present invention scan and aggregate data of bulk historic patient data corresponding to each patient. Embodiments of the present invention utilize a machine learning algorithm to classify each patient. Additional embodiments of the present invention utilize a classification of each patient to optimize processing segments of bulk historic patient data that is initially loaded into a database of a computing system. Further embodiments of the present invention generate a patient synopsis corresponding to a patient based on the bulk historic patient data.

Some embodiments of the present invention recognize that there are several means of providing a comprehensive historical synopsis of a patient and in a steady state, after a system is implemented, the information for the patient is processed upon arrival. However, embodiments of the present invention recognize that challenges exist in making bulk historic patient data available for initial use by a client when implementing a new system that is processing bulk historic patient data after loading. Additionally, challenges exist predicting which patients will arrive so that corresponding historical data can be processed, based on factors that are easily and rapidly derived from demographic and structured information with low computational cost when the extracted historical information is not yet available. In addition, embodiments of the present invention recognize that conventional methods to process the bulk historic patient data such as data migrations where the bulk historic patient data is processed in reverse chronological order fail to overcome these challenges.

Various embodiments of the present invention can operate to optimize the processing of bulk historic patient data sets at initial use utilizing machine learning techniques. For example, the processing of bulk historic patient data sets can conflict with the normal inbound message and document processing, potentially resulting in system backups. Embodiments of the present invention can operate to prevent system backups and increase computing performance by prioritizing processing of bulk historic patient data sets based on a status of a patient and processed during “off hours”, which does not impede performance of the computing system. For example, the “off hours” primarily consist of the nights, weekends and holidays, outside of normal business hours where the bulk of the patient activity occurs.

Implementation of embodiments of the invention may take a variety of forms, and exemplary implementation details are discussed subsequently with reference to the Figures.

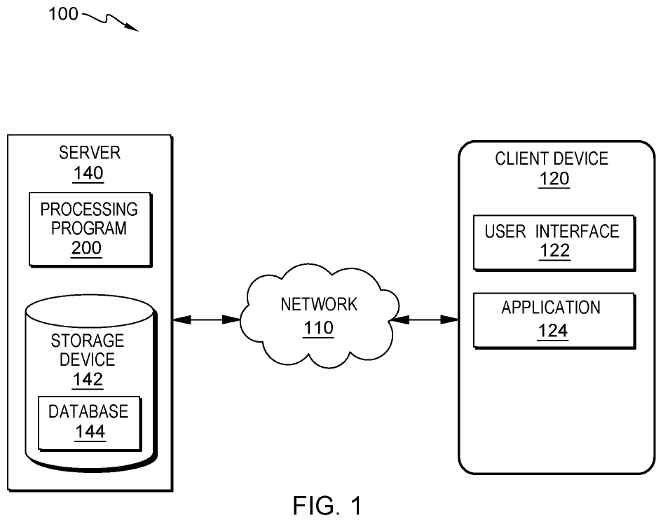

The present invention will now be described in detail with reference to the Figures. FIG. 1 is a functional block diagram illustrating a distributed data processing environment, generally designated 100, in accordance with one embodiment of the present invention. FIG. 1 provides only an illustration of one implementation and does not imply any limitations with regard to the environments in which different embodiments may be implemented. Many modifications to the depicted environment may be made by those skilled in the art without departing from the scope of the invention as recited by the claims.

The present invention may contain various accessible data sources, such as database 144, that may include personal data, content, or information the user wishes not to be processed. Personal data includes personally identifying information or sensitive personal information as well as user information, such as tracking or geolocation information. Processing refers to any, automated or unautomated, operation or set of operations such as collection, recording, organization, structuring, storage, adaptation, alteration, retrieval, consultation, use, disclosure by transmission, dissemination, or otherwise making available, combination, restriction, erasure, or destruction performed on personal data. Processing program 200 enables the authorized and secure processing of personal data. Processing program 200 provides informed consent, with notice of the collection of personal data, allowing the user to opt in or opt out of processing personal data. Consent can take several forms. Opt-in consent can impose on the user to take an affirmative action before personal data is processed. Alternatively, opt-out consent can impose on the user to take an affirmative action to prevent the processing of personal data before personal data is processed. Processing program 200 provides information regarding personal data and the nature (e.g., type, scope, purpose, duration, etc.) of the processing. Processing program 200 provides the user with copies of stored personal data. Processing program 200 allows the correction or completion of incorrect or incomplete personal data. Processing program 200 allows the immediate deletion of personal data.

Distributed data processing environment 100 includes server 140 and client device 120, all interconnected over network 110. Network 110 can be, for example, a telecommunications network, a local area network (LAN), a municipal area network (MAN), a wide area network (WAN), such as the Internet, or a combination of the three, and can include wired, wireless, or fiber optic connections. Network 110 can include one or more wired and/or wireless networks capable of receiving and transmitting data, voice, and/or video signals, including multimedia signals that include voice, data, and video information. In general, network 110 can be any combination of connections and protocols that will support communications between server 140 and client device 120, and other computing devices (not shown) within distributed data processing environment 100.

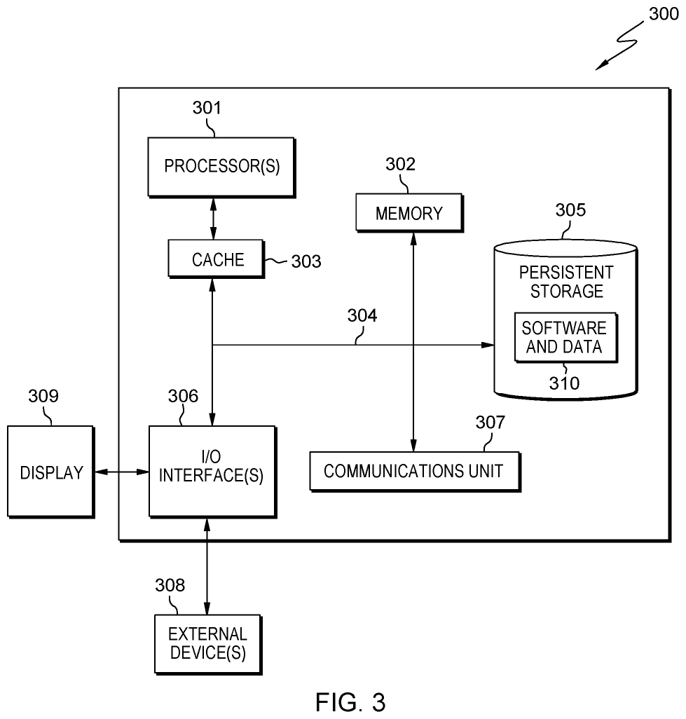

Client device 120 can be one or more of a laptop computer, a tablet computer, a smart phone, smart watch, a smart speaker, virtual assistant, or any programmable electronic device capable of communicating with various components and devices within distributed data processing environment 100, via network 110. In general, client device 120 represents one or more programmable electronic devices or combination of programmable electronic devices capable of executing machine readable program instructions and communicating with other computing devices (not shown) within distributed data processing environment 100 via a network, such as network 110. Client device 120 may include components as depicted and described in further detail with respect to FIG. 3, in accordance with embodiments of the present invention.

Client device 120 includes user interface 122 and application 124. In various embodiments of the present invention, a user interface is a program that provides an interface between a user of a device and a plurality of applications that reside on the client device. A user interface, such as user interface 122, refers to the information (such as graphic, text, and sound) that a program presents to a user, and the control sequences the user employs to control the program. A variety of types of user interfaces exist. In one embodiment, user interface 122 is a graphical user interface. A graphical user interface (GUI) is a type of user interface that allows users to interact with electronic devices, such as a computer keyboard and mouse, through graphical icons and visual indicators, such as secondary notation, as opposed to text-based interfaces, typed command labels, or text navigation. In computing, GUIs were introduced in reaction to the perceived steep learning curve of command-line interfaces which require commands to be typed on the keyboard. The actions in GUIs are often performed through direct manipulation of the graphical elements. In another embodiment, user interface 122 is a script or application programming interface (API).

Application 124 is a computer program designed to run on client device 120. An application frequently serves to provide a user with similar services accessed on personal computers (e.g., web browser, playing music, e-mail program, or other media, etc.). In one embodiment, application 124 is mobile application software. For example, mobile application software, or an “app,” is a computer program designed to run on smart phones, tablet computers and other mobile devices. In another embodiment, application 124 is a web user interface (WUI) and can display text, documents, web browser windows, user options, application interfaces, and instructions for operation, and include the information (such as graphic, text, and sound) that a program presents to a user and the control sequences the user employs to control the program. In another embodiment, application 124 is a client-side application of processing program 200.

In various embodiments of the present invention, server 140 may be a desktop computer, a computer server, or any other computer systems, known in the art. In general, server 140 is representative of any electronic device or combination of electronic devices capable of executing computer readable program instructions. Server 140 may include components as depicted and described in further detail with respect to FIG. 3, in accordance with embodiments of the present invention.

Server 140 can be a standalone computing device, a management server, a web server, a mobile computing device, or any other electronic device or computing system capable of receiving, sending, and processing data. In one embodiment, server 140 can represent a server computing system utilizing multiple computers as a server system, such as in a cloud computing environment. In another embodiment, server 140 can be a laptop computer, a tablet computer, a netbook computer, a personal computer (PC), a desktop computer, a personal digital assistant (PDA), a smart phone, or any programmable electronic device capable of communicating with client device 120 and other computing devices (not shown) within distributed data processing environment 100 via network 110. In another embodiment, server 140 represents a computing system utilizing clustered computers and components (e.g., database server computers, application server computers, etc.) that act as a single pool of seamless resources when accessed within distributed data processing environment 100.

Server 140 includes storage device 142, database 144, and processing program 200. Storage device 142 can be implemented with any type of storage device, for example, persistent storage 305, which is capable of storing data that may be accessed and utilized by client device 120 and server 140, such as a database server, a hard disk drive, or a flash memory. In one embodiment storage device 142 can represent multiple storage devices within server 140. In various embodiments of the present invention, storage device 142 stores numerous types of data which may include database 144. Database 144 may represent one or more organized collections of data stored and accessed from server 140. For example, database 144 includes bulk historic patient data, classifications, etc. In another example, database 144 includes a staging table that includes bulk historic patient data (e.g., flat files) of various storage sources as a result of an extract transform load (ETL). In yet another example, database 144 includes a production table that is utilized to generate a historical patient synopsis in response to request of a user of client device 120. In one embodiment, data processing environment 100 can include additional servers (not shown) that host additional information that accessible via network 110.

Generally, there are several means of providing a comprehensive historical synopsis of a patient and these systems rely on extensive Natural Language Processing (NLP) of the documentation of patient visits. Patient documents can include visit notes, imaging study reports, surgical procedure narratives, treatment plans, and/or other unstructured documents. Additionally, documents can include structured data such as medications, vital signs, allergies, etc. Also, a number of patients are referred to as “frequent fliers” because these patients have multiple chronic conditions (e.g. degenerative disorders, cardiac problems, cancer etc.) that result in deep historical records across many encounters spanning various problems. These patients may have thousands of visits and exams with many thousands of visit notes, reports, orders, labs, etc. and performing NLP, concept extraction, normalization, and additional organization of the patient data for these types of complex patients may take tens of minutes to hours for each patient. Moreover, coupling this with multiple millions of patients with records in a given facility to be processed and the computational workload is staggering.

Furthermore, embodiments of the present invention recognize that the computational workload issue is a “Day 1” type of problem that diminishes over time. For example, preferably the comprehensive data for all arriving patients would be available on the first day of use. However, the processing of complex patient data sets at initial use can overwhelm a computing system, however once processed, the sets would not be re-processed. As a result, there is a decay in a “frequent flier” workload over the first few months and through the first year. In addition, embodiments of the present invention recognize that a significant number of patient records exist that are not needed for a long period of time, which do not need to be processed as the patient may not return for various reasons (e.g., having moved, switched practitioners, deceased, in a healthy state, etc.).

Processing program 200 classifies one or more patients based on a probability that a patient generates a triggering event (e.g., revisit, follow-up, etc.) within a defined time frame to determine a processing order for corresponding historical patient data. In one embodiment, processing program 200 derives factors from demographic and structured information with low computational cost to determine priority processing of one or more segments of bulk historic patient data. For example, in response to a flat file historic patient data (e.g., bulk historic patient data) transfer to staging tables (e.g., database 144) in an ETL, processing program 200 determines one or more segments of the flat file historic patient data to process based on extracted historical information that is not ready for use by the end user application (e.g., application 124, Patient Synopsis generation, etc.) (i.e., the bulk historic patient data needs computationally intensive processing to be ready for the user). Also, the processing of the flat file historic patient data is not the simple data normalization, cleansing, translating, and/or cleansing of various ETL automation tools, as those processes happen as the flat file historic patient data is moved to the staging tables.

For example, processing program 200 can process historical medical records by extracting the medical concepts contained in the textual data via Natural Language Processing (NLP) and building a synopsis or summary based on the historical records of a patient. NLP concept extraction is computationally expensive and provides the summary for the entire set of medical records for each patient, which may be composed of many years of data and hundreds or even thousands of documents which must be processed.

Additionally, processing program 200 can prioritize processing of patient data (e.g., structured data) based on the likelihood of a patient being readmitted, having recurring medical visits or diagnosis, or activities, such as new medications, etc., which processing program 200 can extract from HL7 data or metadata with low computational cost, which indicates the likelihood of follow-up visits. Embodiments of the present invention recognize that prioritized processing is essential for patients with multiple chronic conditions that have frequent visits, which are often referred to as the “frequent fliers.” In addition, processing program 200 can utilize a machine learning (ML) model to correlate the structured data to determine prioritization for processing of medical records for NLP processing and concept extraction. Additionally, processing program 200 enables a systematic “crawl” of patient records for a prioritization process or to process patient records that processing program 200 determines are lower priority.

In another embodiment, processing program 200 queues processing of one or more segments of bulk loaded historical data. For example, processing program 200 determines a plurality of extract features from bulk historical data and utilizes the extracted features to aggregate and filter one or more segments of the bulk historic data to provide a list of data segments can be processed during “off hours.” In this example, processing program 200 uses the extracted features and a machine learning classification algorithm to derive relationships between the selected features and the one or more segments of the bulk historic data. Additionally, the machine learning classification algorithm is utilized to determine a probability of when a data segment of the list of data segments will require processing (i.e., probability of receiving a request to access the data segment).

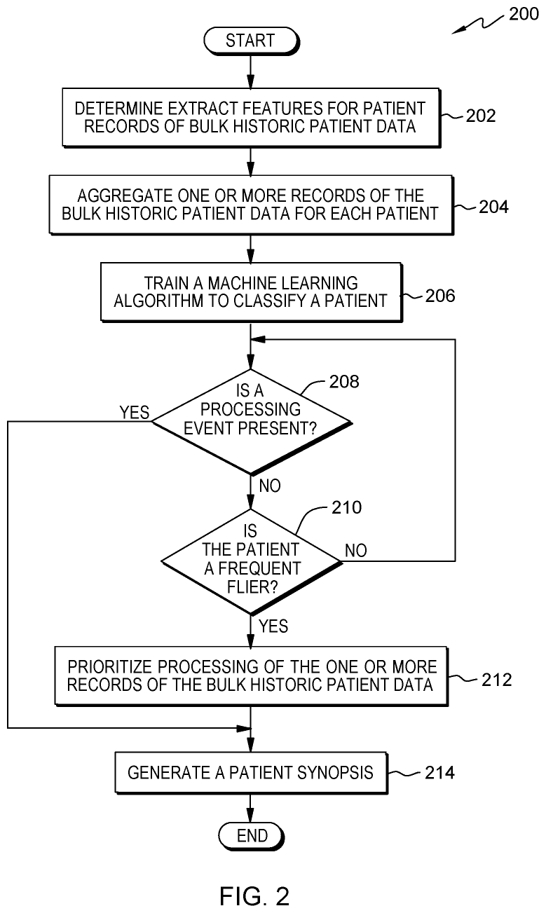

FIG. 2 is a flowchart depicting operational steps of processing program 200, a program that queues bulk historical data of a patient for processing based on a determined priority, in accordance with embodiments of the present invention. In one embodiment, processing program 200 initiates in response to server 140 storing bulk historic patient data in database 144. For example, processing program 200 initiates in response to a user registering (e.g., opting-in) with processing program 200 and transferring flat file historic data to a database of a remote server (e.g., server 140). In another embodiment, processing program 200 is a background application that continuously monitors client device 120 for events corresponding to bulk historic patient data. For example, processing program 200 monitors a computing device (e.g., client device 120) of a user for queries for flat file historic data.

In step 202, processing program 200 determines extract features for patient records of bulk historic patient data. Various embodiments of the present invention recognize that methods exist that seek to predict readmissions and/or clinical encounters based on visit notes and other documents. However, those methods resolve a different problem as those methods are relying on data that has already been processed. For example, processing program 200 identifies one or more segments of unprocessed bulk historic patient data corresponding to one or more patients that are probable to return to a medical setting within a defined timeframe.

In one embodiment, processing program 200 identifies one or more features of incoming data corresponding to queries (e.g., patient demographic query (PDQ), patient identifier cross-referencing (PIX), etc.) of a user of client device 120. For example, processing program 200 utilizes an incoming data feed that includes health level seven (7) (HL7) messages (e.g., patient administration (ADT), orders (ORMs), results (ORUs), charges (DFTs)), which are hierarchical structures associated with a trigger event (e.g., an event in the real world of health care that creates the need for data to flow among systems), to identify features and metadata that can be utilized to aggregate and filter records (e.g., bulk historic patient data) of patients to provide a list of patients whose records should be processed during “off hours” to generate a synopsis report prior to arrival of the patients. In this example, the features and metadata can include demographics such as age, sex, number and frequency of visits, pregnancy status, medication changes, lab results, medical conditions, patient status (e.g., deceased or alive), inpatient status, etc. Also, the features and metadata can include list associated with other structured and unstructured data such as fractures, change in the length of problem list, substance abuse indicators, mental health issues, recent trauma, diagnosis, etc. Additionally, processing program 200 can extract the features and metadata at low computational costs due to information typically being encoded so that natural language processing (NLP) extraction is not required.

In another embodiment, processing program 200 determines a variable importance of the one or more features of the incoming data of communications of the user of client device 120. For example, processing program 200 utilizes feature/variable importance plot techniques to identify the most important features for creation of a dataset for training and prediction of a machine learning algorithm to determine a status of patients. In this example, processing program 200 utilizes Gini Importance or Mean Decrease in Impurity (MDI) to calculate each feature importance as the sum over the number of splits (across all trees) that include the feature, proportionally to the number of samples the feature splits, resulting in a list of the most significant variables in descending order by a mean decrease in Gini. Additionally, processing program 200 can utilizes the top features, which contribute more to the machine learning model than the bottom features as the top features (e.g., above a threshold value) have high predictive power in classifying patients. By contrast, processing program 200 can omit features with low importance, which making the machine learning model simpler and faster to fit and predict.

In step 204, processing program 200 aggregates one or more records of the bulk historic patient data for each patient. In one embodiment, processing program 200 aggregates one or more segments of bulk historic patient data of database 144. For example, processing program 200 logically scans one or more staging tables (e.g., database 144) that include a plurality of patient records (e.g., flat files, electronic medical records (EMR), bulk historic patient data, etc.) to identify records that correspond to each patient. In this example, processing program 200 aggregates identified records of each patient utilizing extracted metadata and features as discussed in step 202.

In step 206, processing program 200 trains a machine learning algorithm to classify a patient. Various embodiments of the present invention train a machine learning algorithm (e.g., linear classifier, nearest neighbor, support vector machines, decision trees, random forest, artificial neural network) to determine whether a person classifies as a “frequent flier,” which are patients with multiple chronic conditions (e.g. degenerative disorders, cardiac problems, cancer, etc.) that have deep historical records across many encounters and various problems (i.e., these patients may have thousands of visits and exams with many thousands of visit notes, reports, orders, labs, etc.). Classification is the process of predicting a class of given data points, where classification predictive models approximate a mapping function (ƒ) from input variables (X) to discrete output variables (y). For example, the current problem is a binary classification problem as there are only two (2) classes: “Frequent Flier” and “not Frequent Flier”.

In one embodiment, processing program 200 utilizes selected features to train a machine learning algorithm to classify a patient of bulk historic patient data of database 144. For example, processing program 200 can utilize a random subspace method (e.g., attribute bagging, feature bagging) to train a random forest classifier to recognize correlations of set of input variables (e.g. selected features of step 202) to discrete output variables (e.g., classifications, statuses, etc.). In this example, a random forest operates by constructing a multitude of decision trees at training time and outputting a class that is the mode of the classes (e.g., classification) or mean prediction (e.g., regression) of the individual trees. Also, random decision forests compensate for decision trees over fitting to corresponding training sets. In addition, processing program 200 compares a set of input variables to a set of criteria that can consider return visit events and imaging orders, which can indicate accuracy as imaging orders are viewed as a high priority for use of a patient synopsis, to identify a default (e.g., “Frequent Flier”) or non-default (e.g., “not Frequent Flier”) status (e.g., classification) of the patient. Alternatively, processing program 200 can utilize other events that correspond to HL7 messages for accuracy and comparison as well.

In another example, once a machine learning algorithm is deployed within a computing system of a user, processing program 200 can augment a model of the machine learning algorithm with additional site specific training data based on return visits and orders in the first 10-30 days of use. As a result, processing program 200 can provide additional weighting for sets of criteria corresponding to patient treatment specialties (e.g. cancer care, joint replacements, transplants, etc.) that are prevalent at that center.

In decision step 208, processing program 200 determines whether a processing event is present. Various embodiments of the present invention utilize several event-based triggers (e.g., admission, scheduled office visits, orders for imaging studies, etc.) to initiate processing of bulk historic patient data. Generally, 80% of HL7 message traffic corresponding to EMR occurs in the normal business day (e.g., ten (10) hours per day five (5) days per week). Also, the processing of these complex patients with deep record sets will conflict with the normal inbound message and document processing, potentially resulting in system backups and often not meeting the need of producing a patient synopsis available for a first visit of the patient. However, in some EMR systems, trigger events do not initiate until a patient arrives for a visit or procedure. One of ordinary skill in the art would appreciate that performing processing of bulk historical patient data prior to admission of a patient and/or in the “off hours” (e.g., nights and weekends where the primary workload is light) can operate to optimize utilization of processing resources of a computing system.

In one embodiment, processing program 200 identifies one or more events that initiate processing of one or more segments of bulk historic patient data corresponding to a patient. For example, processing program 200 utilizes an output of a default classification (e.g., “Frequent Flier”) of a random forest classifier (e.g., machine learning algorithm) as a processing event trigger due to the default classification resulting in one or more records of a patient being promoted in staging tables (e.g., database 144) as discussed below in step 212.

In another example, processing program 200 utilizes metadata (e.g., message types) of HL7 messages to identify events such as ADTs, ORMs, ORUs, etc., corresponding to the messages that indicate a patient visit is imminent within a defined time period. In yet another example, processing program 200 monitors a computing device (e.g., client device 120) of a user to detect when the user opens a patient record (e.g., on demand event) or transmits a PDQ to an enterprise master patient index (EMPI) and desires to see a patient synopsis. Generally, event based triggering of processing of bulk historic data of a given patient is based on an HL7 event, which can conflict in many cases by causing the bulk historic data to be ingested and processed during normal hours exacerbating peak load requirements.

In another embodiment, processing program 200 determines whether an event is present that initiates server 140 to process bulk historic patient data. For example, detects one or more events that initiate processing of one or more segments of bulk historic patient data corresponding to a patient. In one scenario, in demand-based triggering, a user opening a patient record intending to view a historical patient synopsis can lead to a delay in the historical patient synopsis being available (e.g., minutes to hours). In this scenario, given the practice where the patient steps through multiple stages of care/analysis, there may be time to summarize some portion of the record of the patient and make the summary available to the users later in the chain of stages of care/analysis (i.e., demand-based processing is least favorable due to the time required to process the records of a patient (e.g., minutes to hours for extremely complex patients with large historical record sets)).

In another embodiment, if processing program 200 determines that an event is present that initiates processing of bulk historic patient data (decision step 210 “YES” branch), then processing program 200 generates a summary of bulk historic patient data corresponding to a patient as discussed in step 214. For example, if processing program 200 monitors communications of a computing device (e.g., client device 120) of a user and detects that the user transmits a PDQ (e.g., on demand event) to an enterprise master patient index (EMPI) (e.g., application 124, database 144) to open a patient record, then processing program 200 promotes one or more records corresponding to the patient in a staging table (e.g., database 144) for processing to generate a corresponding patient synopsis.

In another embodiment, if processing program 200 determines that an event is not present that initiates processing of bulk historic patient data (decision step 210 “NO” branch), then processing program 200 determine whether aggregated data of database 144 associated with one or more segments of bulk historic patient data corresponding to a patient meet a set of criteria of a classification. For example, if processing program 200 monitors communications of a computing device (e.g., client device 120) of a user and does not determine that the communications include a PDQ (e.g., demand trigger), ADTs, ORMs, and/or ORUs, etc. (e.g., event triggers), then processing program 200 determines whether a default or non-default classification applies to a patient based on one or more records of flat files (e.g., bulk historic patient data) corresponding to the patient.

In decision step 210, processing program 200 determines whether the patient is a frequent flier. In one embodiment, processing program 200 utilizes a machine learning algorithm to determine whether aggregated data of database 144 associated with one or more segments of bulk historic patient data corresponding to a patient meet a set of criteria of a classification. For example, processing program 200 utilizes a random forest classifier (e.g., machine learning algorithm) to determine whether demographics and lists of one or more records of flat files (e.g., bulk historic patient data) corresponding to a patient satisfy a set of criteria of a “Frequent Flier” or “not Frequent Flier” status (e.g., default or non-default classifications). In this example, the set of criteria are dynamic values that indicate a patient is likely to return to a medical setting frequently or within a period of time that may be defined by therapy or additional testing.

In another embodiment, if processing program 200 determines that aggregated data of database 144 associated with one or more segments of bulk historic patient data corresponding to a patient satisfy a set of criteria of a non-default classification (decision step 210 “NO” branch), then processing program 200 can assign the patient a status that affects processing of the one or more segments of bulk historic patient data in a queue of database 144. Additionally, processing program 200 can continuously monitor a communication feed of client device 120 to detect patient queries corresponding to the one or more segments of bulk historic patient data of the patient.

For example, processing program 200 inputs into a random forest classifier, textual data of flat files (e.g., bulk historic patient data) corresponding to demographics and lists (e.g., aggregated features and metadata) of a patient that label the patient as a twenty-five (25) years old, with a frequency of visits below a preset threshold, with no pre-existing or chronic conditions, and no list data. In this example, processing program 200 can receive output of a non-default classification, which indicates that the patient is not likely to return to a medical setting within a defined period of time of time corresponding to an initial data processing period of a computing system. As a result, processing program 200 assigns the patient a “not Frequent Flier” status. In addition, processing program 200 does not promote the flat files of the patient in staging tables of a database (e.g., database 144) for processing and monitors a communications a computing device (e.g., client device 120) of a user for HL7 messages that can trigger processing.

In an alternative scenario, if processing program 200 inputs demographics that include a patient status that indicates a patient is deceased, and the machine learning algorithm returns a non-default classification then, processing program 200 can flag the flat files of the patient as do not process items due to a low probability that a record of the patient will need to be accessed during the initial data processing period. Conversely, the deceased patient may still need to be accessed for clinical studies, epidemiological research, and/or statistical reporting. For these reasons, processing program 200 can assign the records of the deceased patient a variable priority (e.g., low compared to living patients).

In another embodiment, if processing program 200 determines that aggregated data of database 144 associated with one or more segments of bulk historic patient data corresponding to a patient satisfy a set of criteria of a default classification (decision step 210 “YES” branch), then processing program 200 can promote the one or more segments of bulk historic patient data for processing of in a queue of database 144.

For example, processing program 200 inputs into a random forest classifier, textual data of flat files (e.g., bulk historic patient data) corresponding to demographics and lists (e.g., aggregated features and metadata) of a patient that label the patient as twenty-five (25) years old, with a frequency of visits above a preset threshold, recent changes in medication dosage, and list data includes a cancer diagnosis. In this example, processing program 200 can receive output of a default classification, which indicates that the patient is likely to return to a medical setting within a defined period of time corresponding to an initial data processing period of a computing system. As a result, processing program 200 assigns the patient a “Frequent Flier” status and promotes the flat files of the patient in the queue of staging tables of a database (e.g., database 144) for processing.

In step 212, processing program 200 prioritizes processing of the one or more records of the bulk historic patient data. Generally, a crawler scans a system for patients with records to ingest and the scan can be ordered based on a variety of factors (e.g. reverse chronological order for most recent event for that patient, simple patient identity order, etc.) The crawler can typically be set up to operate with maximum load/speed in the “off hours” and be throttled back or stopped during normal business hours, which is a typical data migration pattern used when replacing one system with another. However, various embodiments of the present invention can assign the crawler an additional parameter to only ingest records of patients that are not marked as deceased by processing program 200.

In one embodiment, processing program 200 promotes one or more segments of bulk historic patient data to database 144 using an assigned classification. For example, processing program 200 generates a list including one or more records of flat files (e.g., bulk historic patient data) corresponding to each of one or more patients that are assigned a “Frequent Flier” status. In this example, processing program 200 modifies an order of a staging table (e.g., database 144) so that the one or more records corresponding to the generated list are promoted to a production table (e.g., database 144) for processing prior to patient records associated with “not-Frequent Flier” status (i.e., patients meeting the frequent flier criteria queued for processing).

In another example, processing program 200 assigns a rank to one or more records of flat files (e.g., bulk historic patient data) that have a “Frequent Flier” status. In this example, processing program 200 assigns the rank to a patient record of the one or more records based on a probability of a patient returning to a medical setting within a defined time period. In one scenario, if two patients are assigned a “Frequent Flier” status, processing program 200 would utilize data (e.g., ADTs, ORMs, etc.) corresponding to the patients to determine which patient is likely to return before the other and assign a rank accordingly.

Additionally, processing program 200 can assign a processing method to the one or more segments of bulk historic patient data of database 144 using a triggering event. For example, processing program 200 can assign different levels of priority for processing of patient records. In this example, processing program 200 generates a priority order for processing of text analytics and summarization by a computing system, where tasks corresponding to live incoming data, on demand events, HL7 triggered, frequent fliers status, crawler (e.g., default) are accordingly ranked from highest to lowest priority.

In another scenario, if all the triggering events occur and initiate processing of bulk historic patient data without proper prioritization, background operations (e.g., crawler and frequent flier processing) can overwhelm a computing system and hinder live data processing. Additionally, at some point in time after initiating processing program 200 the background processing for “Frequent Flier” and crawler may be halted where processing program 200 determines the remaining patients records in the staging tables (e.g., database 144) have data that is below a certain threshold and if processed on one of a HL7 event trigger would not pose a significant additional workload on a computing system.

In step 214, processing program 200 generates a patient synopsis. In one embodiment, processing program 200 generates a summary of one or more segments of bulk historic patient data of database 144 corresponding to a patient. For example, processing program 200 promotes one or more records of a patient from a staging table to a production table and determines whether the one or more records include multiple identities based on a list within the ADT messages of an identity set in the bulk historic patient data. In this example, processing program 200 reconciles multiple identities and extracts concepts from the one or more records using NLP techniques. Additionally, processing program 200 utilizes the extracted concepts and natural language generation (NLG) to generate a summary of the textual data corresponding to each of the extracted concepts of the one or more records.

In one scenario, processing program 200 identifies contextually relevant information (e.g., insight) of text and image data (e.g., structured and unstructured data, medical imaging data, etc.) using concepts corresponding to extracted features. Also, processing program 200 aggregates and displays the contextually relevant information of the text and image data to a user. Additionally, processing program 200 can generate a textual summary of the contextually relevant information utilizing volumes of text and image data (i.e., extracting relevant information from bulk historical data and displaying the bulk historic data in a single-view summary with a picture archiving and communications system (PACS)).

FIG. 3 depicts a block diagram of components of client device 120 and server 140, in accordance with an illustrative embodiment of the present invention. It should be appreciated that FIG. 3 provides only an illustration of one implementation and does not imply any limitations with regard to the environments in which different embodiments may be implemented. Many modifications to the depicted environment may be made.

FIG. 3 includes processor(s) 301, cache 303, memory 302, persistent storage 305, communications unit 307, input/output (I/O) interface(s) 306, and communications fabric 304. Communications fabric 304 provides communications between cache 303, memory 302, persistent storage 305, communications unit 307, and input/output (I/O) interface(s) 306. Communications fabric 304 can be implemented with any architecture designed for passing data and/or control information between processors (such as microprocessors, communications and network processors, etc.), system memory, peripheral devices, and any other hardware components within a system. For example, communications fabric 304 can be implemented with one or more buses or a crossbar switch.

Memory 302 and persistent storage 305 are computer readable storage media. In this embodiment, memory 302 includes random access memory (RAM). In general, memory 302 can include any suitable volatile or non-volatile computer readable storage media. Cache 303 is a fast memory that enhances the performance of processor(s) 301 by holding recently accessed data, and data near recently accessed data, from memory 302.

Program instructions and data (e.g., software and data 310) used to practice embodiments of the present invention may be stored in persistent storage 305 and in memory 302 for execution by one or more of the respective processor(s) 301 via cache 303. In an embodiment, persistent storage 305 includes a magnetic hard disk drive. Alternatively, or in addition to a magnetic hard disk drive, persistent storage 305 can include a solid state hard drive, a semiconductor storage device, a read-only memory (ROM), an erasable programmable read-only memory (EPROM), a flash memory, or any other computer readable storage media that is capable of storing program instructions or digital information.

The media used by persistent storage 305 may also be removable. For example, a removable hard drive may be used for persistent storage 305. Other examples include optical and magnetic disks, thumb drives, and smart cards that are inserted into a drive for transfer onto another computer readable storage medium that is also part of persistent storage 305. Software and data 310 can be stored in persistent storage 305 for access and/or execution by one or more of the respective processor(s) 301 via cache 303. With respect to client device 120, software and data 310 includes data of user interface 122 and application 124. With respect to server 140, software and data 310 includes data of storage device 142 and processing program 200.

Communications unit 307, in these examples, provides for communications with other data processing systems or devices. In these examples, communications unit 307 includes one or more network interface cards. Communications unit 307 may provide communications through the use of either or both physical and wireless communications links. Program instructions and data (e.g., software and data 310) used to practice embodiments of the present invention may be downloaded to persistent storage 305 through communications unit 307.

I/O interface(s) 306 allows for input and output of data with other devices that may be connected to each computer system. For example, I/O interface(s) 306 may provide a connection to external device(s) 308, such as a keyboard, a keypad, a touch screen, and/or some other suitable input device. External device(s) 308 can also include portable computer readable storage media, such as, for example, thumb drives, portable optical or magnetic disks, and memory cards. Program instructions and data (e.g., software and data 310) used to practice embodiments of the present invention can be stored on such portable computer readable storage media and can be loaded onto persistent storage 305 via I/O interface(s) 306. I/O interface(s) 306 also connect to display 309.

Display 309 provides a mechanism to display data to a user and may be, for example, a computer monitor.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, python, or the like, and procedural programming languages, such as the “C” programming language or similar programming languages or machine learning computational frameworks such as TensorFlow, PyTorch or others. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A method for processing bulk historical data, the method comprising:
identifying, by one or more processors, one or more features of messages of incoming data queries of a computing device, wherein the one or more features include structured and unstructured data;
aggregating, by one or more processors, one or more segments of bulk historic data for each individual of a plurality of individuals based at least in part on the one or more features of the messages of the incoming data queries;
determining, by one or more processors, a classification of each individual of the plurality of individuals based at least in part on the aggregated one or more segments of the bulk historic data; and
prioritizing, by one or more processors, processing of the aggregated one or more segments of the bulk historic data based at least in part on the classification of each individual of the plurality of individuals.

2. The method of claim 1, further comprising:
creating, by one or more processors, one or more training sets based on the one or more features of the messages of the incoming data queries and set of criteria of two classes, wherein the two classes corresponding to a status of an individual;
creating, by one or more processors, one or more testing sets based on the one or more features of the messages of the incoming data queries and the set of criteria of the two classes; and
training, by one or more processors, a machine learning algorithm utilizing the one or more created training sets and testing sets.

3. The method of claim 1, further comprising:
extracting, by one or more processors, textual data corresponding to one or more concepts of the aggregated one or more segments of the bulk historic data of an individual of the plurality of individuals; and
generating, by one or more processors, a summary of the aggregated one or more segments of the bulk historic data of the individual of the plurality of individuals based at least in part on the textual data corresponding to each of the extracted concepts.

4. The method of claim 3, further comprising:
identifying, by one or more processors, one or more triggering events that initiate processing of the aggregated one or more segments of bulk historic data corresponding to the individual of the plurality of individuals.

5. The method of claim 1, wherein prioritizing processing of the aggregated one or more segments of the bulk historic data based at least in part on the classification of each individual of the plurality of individuals, further comprises:
generating, by one or more processors, a list corresponding to a processing order of the aggregated one or more segments of the bulk historic data based at least in part on a triggering event;
assigning, by one or more processors, a rank to one or more individuals of the plurality of individuals based at least in part on a probability of receiving a request to access the aggregated one or more segments of bulk historic data corresponding to the individual within a defined time period; and
modifying, by one or more processors, the list corresponding to the processing order based on the assigned rank of the one or more individuals.

6. The method of claim 1, wherein identifying the one or more features of messages of incoming data queries of the computing device, further comprises:
determining, by one or more processors, a variable importance of each of the one or more features of messages of incoming data queries of the computing device; and
selecting, by one or more processors, features of messages of incoming data queries of the computing device above a threshold value.

7. The method of claim 1, wherein the one or more features include demographics and patient information of structured and unstructured data and the bulk historic data is medical data of a patient.

8. A computer program product for processing bulk historical data, the computer program product comprising:
one or more computer readable storage media and program instructions stored on the one or more computer readable storage media, the program instructions comprising:
program instructions to identify one or more features of messages of incoming data queries of a computing device, wherein the one or more features include structured and unstructured data;
program instructions to aggregate one or more segments of bulk historic data for each individual of a plurality of individuals based at least in part on the one or more features of the messages of the incoming data queries;
program instructions to determine a classification of each individual of the plurality of individuals based at least in part on the aggregated one or more segments of the bulk historic data; and
program instructions to prioritize processing of the aggregated one or more segments of the bulk historic data based at least in part on the classification of each individual of the plurality of individuals.

9. The computer program product of claim 8, further comprising program instructions, stored on the one or more computer readable storage media, to:
create one or more training sets based on the one or more features of the messages of the incoming data queries and set of criteria of two classes, wherein the two classes corresponding to a status of an individual;
create one or more testing sets based on the one or more features of the messages of the incoming data queries and the set of criteria of the two classes; and
train a machine learning algorithm utilizing the one or more created training sets and testing sets.

10. The computer program product of claim 8, further comprising program instructions, stored on the one or more computer readable storage media, to:
extract textual data corresponding to one or more concepts of the aggregated one or more segments of the bulk historic data of an individual of the plurality of individuals; and
generate a summary of the aggregated one or more segments of the bulk historic data of the individual of the plurality of individuals based at least in part on the textual data corresponding to each of the extracted concepts.

11. The computer program product of claim 10, further comprising program instructions, stored on the one or more computer readable storage media, to:
identify one or more triggering events that initiate processing of the aggregated one or more segments of bulk historic data corresponding to the individual of the plurality of individuals.

12. The computer program product of claim 8, wherein the program instructions to prioritize processing of the aggregated one or more segments of the bulk historic data based at least in part on the classification of each individual of the plurality of individuals, further comprise program instructions to:
generate a list corresponding to a processing order of the aggregated one or more segments of the bulk historic data based at least in part on a triggering event;
assign a rank to one or more individuals of the plurality of individuals based at least in part on a probability of receiving a request to access the aggregated one or more segments of bulk historic data corresponding to the individual within a defined time period; and
modify the list corresponding to the processing order based on the assigned rank of the one or more individuals.

13. The computer program product of claim 8, wherein the program instructions to identify the one or more features of messages of incoming data queries of the computing device, further comprise program instructions to:
determine a variable importance of each of the one or more features of messages of incoming data queries of the computing device; and
select features of messages of incoming data queries of the computing device above a threshold value.

14. The computer program product of claim 8, wherein the one or more features include demographics and patient information of structured and unstructured data and the bulk historic data is medical data of a patient.

15. A computer system for processing bulk historical data, the computer system comprising:
one or more computer processors;
one or more computer readable storage media; and
program instructions stored on the computer readable storage media for execution by at least one of the one or more processors, the program instructions comprising:
program instructions to identify one or more features of messages of incoming data queries of a computing device, wherein the one or more features include structured and unstructured data;
program instructions to aggregate one or more segments of bulk historic data for each individual of a plurality of individuals based at least in part on the one or more features of the messages of the incoming data queries;
program instructions to determine a classification of each individual of the plurality of individuals based at least in part on the aggregated one or more segments of the bulk historic data; and
program instructions to prioritize processing of the aggregated one or more segments of the bulk historic data based at least in part on the classification of each individual of the plurality of individuals.

16. The computer system of claim 15, further comprising program instructions, stored on the one or more computer readable storage media, to:
create one or more training sets based on the one or more features of the messages of the incoming data queries and set of criteria of two classes, wherein the two classes corresponding to a status of an individual;
create one or more testing sets based on the one or more features of the messages of the incoming data queries and the set of criteria of the two classes; and
train a machine learning algorithm utilizing the one or more created training sets and testing sets.

17. The computer system of claim 15, further comprising program instructions, stored on the one or more computer readable storage media, to:
extract textual data corresponding to one or more concepts of the aggregated one or more segments of the bulk historic data of an individual of the plurality of individuals; and
generate a summary of the aggregated one or more segments of the bulk historic data of the individual of the plurality of individuals based at least in part on the textual data corresponding to each of the extracted concepts.

18. The computer system of claim 17, further comprising program instructions, stored on the one or more computer readable storage media, to:
identify one or more triggering events that initiate processing of the aggregated one or more segments of bulk historic data corresponding to the individual of the plurality of individuals.

19. The computer system of claim 15, wherein the program instructions to prioritize processing of the aggregated one or more segments of the bulk historic data based at least in part on the classification of each individual of the plurality of individuals, further comprise program instructions to:
generate a list corresponding to a processing order of the aggregated one or more segments of the bulk historic data based at least in part on a triggering event;
assign a rank to one or more individuals of the plurality of individuals based at least in part on a probability of receiving a request to access the aggregated one or more segments of bulk historic data corresponding to the individual within a defined time period; and
modify the list corresponding to the processing order based on the assigned rank of the one or more individuals.

20. The computer system of claim 15, wherein the program instructions to identify the one or more features of messages of incoming data queries of the computing device, further comprise program instructions to:
determine a variable importance of each of the one or more features of messages of incoming data queries of the computing device; and
select features of messages of incoming data queries of the computing device above a threshold value.
